(19) United States
(12) Patent Application Publication
(10) Pub. No.: US 2006/0127457 A1
(43) Pub. Date: Jun. 15, 2006

(54) Fabrics impregnated with antimicrobial agents

(76) Inventor: Gilbert Buchalter, Millburn, NJ (US)

Correspondence Address: RICHARD R. MUCCINO, 758 SPRINGFIELD AVENUE, SUMMIT, NJ, 07901, US

(21) Appl. No.: 11/010,098

(22) Filed: Dec. 10, 2004

(51) Int. Cl. A61K 9/70; B32B 27/04

(52) U.S. Cl. 424/443; 442/128

(57) ABSTRACT

The present invention provides an antimicrobial fabric comprising a fibrous material treated with an antimicrobially effective amount of an antimicrobial agent. The present invention further provides a method for preparing an antimicrobial fabric that exhibits antimicrobial properties comprising admixing an antimicrobially effective amount of an antimicrobial agent with a fibrous material.

### BACKGROUND OF THE INVENTION

1. Field of the Invention

The present invention provides antimicrobial fabrics comprising a fibrous material treated with an antimicrobially effective amount of an antimicrobial agent. The present invention further provides methods for preparing an antimicrobial fabric that exhibits antimicrobial properties comprising admixing an antimicrobially effective amount of an antimicrobial agent with a fibrous material.

2. Description of the Background

Hospital-acquired infections through the transfer of microbial agents are of great concern. Many techniques to sterilize hospital fabrics have been employed to solve this problem. Such sterilization techniques must be effective against bacteria, viruses, fungi, and spores, and the antimicrobial agent must be able to bond to, and be non-reactive with, conventionally used hospital fabrics, be non-toxic, substantially odorless, non-corrosive, non-irritating, and relatively easy to apply.

Many chemical antimicrobial agents generally incorporate heavy metals ions such as zinc or silver. A problem with silver ions is that silver is expensive and when silver ions are exposed to sulfides in the object to be treated, the silver ions tend to form water-insoluble sulfides which stain the treated object.

Accordingly, there is a need for fabrics impregnated with an antimicrobial agent which have a wide disinfecting spectrum.

U.S. Pat. No. 6,726,936 (Asano et al.) discloses antimicrobial agents which include silver-chloro complex salts. (Asano et al. at col. 67, lines 42-51). Asano et al. states that the antimicrobial agents may be used to apply antimicrobial treatment such as disinfecting, sterilizing, deodorizing, mildew proofing, and sanitizing on target objects such as houses, hospitals, public facilities, industrial products, industrial wastes, and home appliances. Asano et al. further states that the invention also relates to detergents and laundry supplements used to wash clothes, bedding, and to medical use products, as well as to disposable sheets. (Asano et al. at col. 1, lines 7-25).

U.S. Pat. No. 6,713,414 (Pomplun et al.) discloses a wet wipe comprising a fibrous material, a binder composition for binding the fibrous material into an integral web, and a wetting composition. Pomplun et al. states that the binder composition comprises a sulfonate anion modified acrylic acid terpolymer and a non-crosslinking poly(ethylene-vinyl acetate); and the binder composition is insoluble in a neutral salt solution containing about 1% comprising monovalent ions and is soluble in water containing up to about 200 ppm of multivalent ions. (Pomplun et al. at col. 64, lines 27-40). Pomplun et al. states that the ion-sensitive polymer formulations of the present invention have a “trigger property” such that the flushable products maintain integrity during storage and use, but disperse after disposal in the toilet. (Pomplun et al. at col. 3, line 36, to col. 4, line 6).

U.S. Pat. No. 6,521,553 (Tabata et al.) discloses a deodorant fibrous material comprising a fibrous material having a surface to which is attached a deodorant composition. Tabata et al. states that the fibrous material may include (1) an inorganic type component (partially or entirely replaceable by a polyorganic acid, ester or salt thereof; (2) a polyvinyl amine compound and/or a hydrazide compound; and (3) a synthetic resin attached to the surface of the fibrous material. (Tabata et al. at col. 1, line 63, to col. 2, line 2) Tabata et al. states that the deodorant fibrous material has a durable deodorizing characteristic for undesirable odors such as ammonium, amines, hydrogen sulfide, mercaptans, aldehydes, and acetic acid contained in tobacco odors. (Tabata et al. at col. 1, lines 55-62). The inorganic type component may be a compound of copper or aluminum. (Tabata et al. at col. 15, lines 1-3).

U.S. Pat. No. 6,517,849 (Seger et al.) discloses an antiviral tissue product comprising a fibrous ply; an antiviral composition comprising a pyrrolidone carboxy acid; and an “Easiness to Loosen in Water” test value of greater than about 100 seconds. (Seger et al. at col. 23, lines 63-67, to col. 24, lines 1-2). Seger et al. states that the antiviral tissue paper comprising pyrrolidonecarboxylic acid has the ability to kill certain strains of viruses which come into contact with the tissue, and in addition to its antiviral efficacy, pyrrolidone carboxylic acid is mild to the skin thus mitigating the potential for skin irritation. (Seger et al. at col. 1, lines 20-30).

U.S. Pat. No. 5,914,120 (Wellinghoff et al.) discloses a composite for retarding, killing, preventing, or controlling microbiological contamination comprising a hydrophilic material containing an alpha-amino ester, alpha-amino alcohol, or alpha-amino ester and a chlorite salt; and a hydrophobic material containing an acid releasing agent. The hydrophilic and hydrophobic materials are adjacent and substantially free of water. The hydrophilic material is capable of generating and releasing chlorine dioxide after hydrolysis of the acid releasing agent. (Wellinghoff et al. at col. 51, line 66, to col. 52, line 8.) Wellinghoff et al. states that the biocidal composition provides sustained release of chlorine dioxide and provides a composite including an amine-containing hydrophilic material having improved long term storage stability for retarding, controlling, killing or preventing microbiological contamination (e.g., bacteria, fungi, viruses, mold spores, algae, and protozoa), deodorizing and/or retarding. preventing or controlling chemotaxis by release of chlorine dioxide. (Wellinghoff et al. at col. 1, lines 10-19)

U.S. Pat. No. 5,888,526 (Tsubai et al.) Tsubai et al. reference discloses a fibrous material containing an antibacterial and/or antifungal agent comprising a metal salt of an organic compound wherein the metal salt is a complex of a silver salt, a copper salt, and a zinc salt. (Tsubai et al. at col. 32, lines 29-32).

U.S. Pat. No. 5,833,970 (Cox) discloses a deodorant for abating odors, other than human body odors. The deodorant consists of aluminum water-soluble inorganic salt, 5% to 90%, of an additional water-soluble inorganic salt of metal selected from the group consisting of iron, copper, zinc and nickel but less than the amount of aluminum water-soluble inorganic salt, at least 2%, and glycol 3% to 60%; and having a pH less than 5 or greater than 9 in the presence of water. (Cox at col. 6, lines 20-39).

U.S. Pat. No. 5,358,919 (Wu) discloses a method of preparing a composition of matter consisting of (1) impregnating alumina having a surface area of at least about 40 m2/g with a solution of at least one metal salt selected from the group consisting of copper(II) sulfate, iron(II) sulfate, cobalt(II) sulfate, nickel(II) sulfate, manganese(II) sulfate, zinc sulfate and magnesium sulfate; (2) calcining the material obtained in step (1) for a period of at least 0.5 hour at a temperature of about 400°-750° C.; (3) heating for a period of at least one hour, at a temperature in the range of about 40° C. to about 90° C. and in substantial absence of water, a mixture consisting essentially of the calcined material obtained in step (2), aluminum chloride and at least one chlorinated hydrocarbon selected from the group consisting of dichloromethane, chloroform, carbon tetrachloride, 1,1-dichloroethane, 1,2-dichloroethane, 1,1,1-trichloroethane, 1,1-dichloropropane, 2,2-dichloropropane, 1-chlorobutane and 2-chloro-2-methylbutane, wherein the weight ratio of AlCl₃ to the calcined material obtained in step (2) is at least about 0.35:1; and (4) separating the solid material contained in the reaction mixture formed in step (3) from the chlorinated hydrocarbon under a dry gas atmosphere. (Wu at col. 15, lines 8-38).

U.S. Pat. No. 4,521,530 (Zackay et al.) discloses an oxidation catalyst consisting of palladium, nickel, and copper on a substrate. (Zackay et al. at col. 5, lines 18-21). The alumina substrate may be impregnated with a halide salt solution of copper chloride and copper sulfate. Zackay et al. at col. 2, lines 25-58).

U.S. Pat. No. 4,283,386 (Van Scott et al.) discloses a method for hair grooming comprising topically applying to the hair a cosmetically effective amount of a composition containing from about 0.2 to 10% of a compound selected from the group consisting of cysteic acid, cysteine sulfinic acid and homocysteic acid and chelates of the compound with a metallic compound selected from the group consisting of ferric chloride, copper sulfate, copper carbonate, zinc sulfate, zinc oxide, zinc chloride, aluminum chlorohydrate, and aluminum zinc sulfate. (Van Scott et al. at col. 8, lines 4-14)

United States patent applic. pub. No. 2003/0159200 (Elrod) discloses a method of making antimicrobial fabrics comprising the steps of creating a free radical species on a surface of the fabric; and reacting a polymerizable monomer with the free radical species to initiate graft polymerization of the monomer on the fabric surface. The monomer has a functional group selected from antimicrobial groups and precursors to antimicrobial groups. (Elrod at col. 4)

PCT patent application WO pub. No. WO 02/45953 (Milliken) discloses a spandex yarn comprising an antimicrobial compound in discrete areas of the yarn wherein some antimicrobial compound is present at the surface of the yarn and some antimicrobial is present within the area of the yarn from the center to a distance of one-half the radius of the yarn. (Milliken at col. 12) The antimicrobial compound is a silver compound. (Milliken at col. 1, first paragraph)

PCT patent application WO pub. No. WO 02/092890 (Creavis) discloses the use of antimicrobial polymers for producing fibres and fabric with an antimicrobial action, and to the use of the antimicrobial fibres and fabric. (Creavis at abstract)

PCT patent application WO pub. No. WO 01/00252 (Zakryto) discloses the prevention of inflammatory infectious diseases caused by the action of microbiological factors by a medical fabric which has virucidal and antimicrobial activity. The medical fabric, made from natural tissues, contains as an antimicrobial substance a quaternary ammonium base with 10 to 18 carbon atoms, Catamin AB, and an alcohol with multiple atoms. (Zakryto at abstract)

PCT patent application WO pub. No. WO 03/01650 (Czuczak) discloses that partially halogenated hydantoins, such as monochlorodimethyl hydantoin, sanitize textiles with minimal color fading and textile degradation. (Czuczak at abstract)

Japanese patent application No. 10139908 (Ykk Corp) discloses a method for producing an antibacterial and antifungal synthetic resin material and for providing synthetic resin articles having persistent antibacterial and antifungal effect. Ykk Corp describes the preparation of a synthetic resin substrate containing an additive which reacts with a metal salt to form an antibacterial and antifungal substance and part of which is exposed to the surface. The part of the additive which is exposed to the surface reacts with a metal salt to form an antibacterial and antifungal substance. Examples of the additives used include oxides, hydroxides, and carbonates of aluminum, magnesium and calcium. Examples of the metal salts used include chlorides, sulfates, acetates, nitrates and carbonates of copper, zinc, silver and nickel. (Ykk Corp. at abstract)

Japanese patent application No. 61205617 (Minoru) discloses the recovery of powder metallic copper in high yield and useful aluminum as by products by reacting waste liquor of water-soluble copper salts with metallic aluminum. Waste liquor containing a water-soluble salt such as cupric chloride, copper sulfate, is blended with metallic aluminum. (Minoru at abstract)

Japanese patent application No. 58015572 (Daiichi Seimo) discloses a coating agent for cultivating a net to prevent seaweed from proliferating by incorporating a salt of aluminum, copper, zinc, etc. into a water-soluble resin that is used as a base. A salt of aluminum, copper, zinc, e.g., aluminum chloride, copper sulfate, zinc chloride (preferably, the metal being chelated with ethylenediaminetetraacetic acid, citric acid, etc) is added to an aqueous solution of a water-soluble resin, e.g., a partially saponified polyvinyl acetate. This coating agent is said to effectively control the harmful green layer without affecting the growth of useful phorphyra tenera generally called the black layer. (Daiichi Seimo at abstract).

While the above antimicrobial compositions are reported to inhibit the production of microbes, none of the above compositions are entirely satisfactory. None of the antimicrobial compositions has the ability to simultaneously destroy (bacteriocidal) or suppress the growth of (bacteriostatic) microorganisms. The present invention provides such improved antimicrobial fabric comprising without the disadvantages characteristic of previously known compositions. This invention also provides methods for preparing an antimicrobial fabric that exhibits antimicrobial properties comprising admixing an antimicrobially effective amount of an antimicrobial agent to a fibrous material.

### SUMMARY OF THE INVENTION

The present invention provides an antimicrobial fabric comprising a fibrous material treated with an antimicrobially effective amount of an antimicrobial agent.

The present invention further provides a method for preparing an antimicrobial fabric that exhibits antimicrobial properties comprising admixing an antimicrobially effective amount of an antimicrobial agent with a fibrous material.

### DETAILED DESCRIPTION OF THE INVENTION

Applicant has discovered antimicrobial fabrics comprising a fibrous material treated with an antimicrobially effective amount of an antimicrobial agent. Applicant has also discovered methods for preparing an antimicrobial fabric that exhibits antimicrobial properties comprising admixing an antimicrobially effective amount of an antimicrobial agent with a fibrous material.

The antimicrobial fabric comprising a fibrous material treated with an antimicrobial agent may be used as clothing or hospital draping to prevent cross contamination between patients and hospital personnel in contact with infectious material, animate or inanimate. The antimicrobial fabric treated or impregnated with an antimicrobial agent may be a fibrous material that is a woven or a non-woven material, or a disposable or a non-disposable material. The antimicrobial fabric treated or impregnated with the antimicrobial agent preferably is substantive such that the antimicrobial agent confers antimicrobial properties to the fibrous material.

The antimicrobial fabric comprising a fibrous material treated with an antimicrobial agent should be safe for the wearer (non-allergenic) as well as for people in casual contact with the wearer of the fabric.

The antimicrobial agent by itself may act as an indicator to distinguish between fibrous material treated with the antimicrobial agent and fibrous material untreated with the antimicrobial agent. The fibrous material treated with the antimicrobial agent may also be made distinguishable from fibrous material untreated with an antimicrobial agent by addition of an indicator to show treatment.

The fibrous materials which may be employed in the present invention may be selected from a wide variety of fibrous materials. Illustrative, non-limiting examples of fibrous materials, include woven and non-woven materials which may be natural fibers or non-natural fibers, and mixtures thereof. The fibrous material may be a disposable or a non-disposable material, and preferably is a disposable material. The fibrous material should also be a non-allergenic material.

Illustrative, non-limiting examples of natural fibers may be selected from the group consisting of cotton, wool, silk, flax, mohair, linen, hemp, ramie, and jute. Preferable natural fibers maybe selected from the group consisting of cotton, wool, silk, flax, mohair, and linen. More preferably, the natural fibers maybe selected from the group consisting of cotton, wool, silk, and flax. Most preferably, the natural fibers maybe selected from the group consisting of cotton, wool, and silk.

Illustrative, non-limiting examples of non-natural fibers may be selected from the group consisting of rayons, acetates, nylons, polyesters, polyolefins, polyamides, acrylics, modacrylics fibers, fluorocarbons, glass fibers, and spandex. Preferable non-natural fibers maybe selected from the group consisting of rayons, acetates, nylons, polyesters, polyolefins, polyamides, acrylics, modacrylics fibers, and fluorocarbons. More preferably, the non-natural fibers maybe selected from the group consisting of rayons, acetates, nylons, polyesters, polyolefins, polyamides, and acrylics. Most preferably, the non-natural fibers maybe selected from the group consisting of rayons, acetates, nylons, polyesters, and polyolefins.

Preferably, the fibrous material treated with an antimicrobial agent is a medical fabric applicable to medical/hospital use. Such medical fabrics include, but are not limited to, surgical gowns, scrubs, tabards, gloves, socks, hats, belts, shoes, insoles for shoes, padding, bandages, draping, sheeting, pillow cases, towels, rugs, and the like.

The antimicrobial agents which may be employed in the present invention may be selected from a wide variety of antimicrobial agents. Antimicrobial agents are compounds that destroy (bacteriocidal) or suppress the growth of (bacteriostatic) microorganisms. The specific antimicrobial agent is selected based upon the microorganism(s) to be treated; the physical and chemical compatibility of the antimicrobial agent with the system to be treated; the stability of the antimicrobial agent under storage conditions; and toxicity, environmental, and economical factors. The antimicrobial agent should also be a non-allergenic material. The water-solubility of the antimicrobial agent is a desirable factor, but is not necessary. Solvents other than water and suspensions of antimicrobial agents may also be employed. Mixtures of antimicrobial agents, which may or may not have synergistic activity, may also be used

The antimicrobial agent may be an anti-viral agent, an antibacterial agent, an antimycotic agent, or combinations thereof. Illustrative, non-limiting examples of antimicrobial agents, include phenolic compounds; halogen compounds; quaternary ammonium compounds; metal derivatives; amines; alkanolamines; nitro derivatives; anilides; organo-sulfur and sulfur-nitrogen compounds; and the like.

Preferred antimicrobial agents in the present invention are antimicrobial aluminum salts. More preferred antimicrobial aluminum salts are aluminum chlorohydrate, aluminum chlorohydrate-containing compounds, and equivalents thereof. Illustrative non-limiting examples of aluminum chlorohydrate-containing compounds and equivalents thereof include aluminum chlorohydrate, aluminum sesquicholorhydrate and aluminum dichlorohydrate; aluminum zirconium chlorohydrates including aluminum zirconium octachlorohydrate, aluminum zirconium pentachlorohydrate, aluminum zirconium tetrachlorohydrate and aluminum zirconium trichlorohydrate; aluminum chloride hydrate; and other aluminum salts, provided that such other salts are not skin irritants at concentrations suitable for use in the present invention and do not cause other adverse side effects. Also useful in the present invention are partially dehydrated derivatives of the chlorohydrates of aluminum and aluminum zirconium, each complexed with polyalkylene glycol, for example polyethylene glycol or propylene glycol, or in which some of the water of hydration has been replaced by glycine. Furthermore, in the aluminum zirconium chlorohydrates the aluminum to zirconium ratio can be variable. For convenience of reference herein these compounds are referred to as chlorohydrates. Typically, the aluminum chlorohydrate is commercially available or can be prepared by art-recognized procedures from known compounds or intermediates readily available or prepared by known reaction schemes.

Preferably, the antimicrobial aluminum salt is aluminum chlorohydrate. Aluminum chlorohydrate (aluminum chlorhydrate, aluminum chloride hexahydrate, AlCl₃.6H₂O, mol. wt. 241.43) is a disinfectant and a topical astringent.

Other preferred antimicrobial agents in the present invention are antimicrobial copper salts. Illustrative non-limiting examples of antimicrobial copper salts include cupric sulfate (copper(II) sulfate, CuSO₄); cupric acetate (Cu(CH₃COO)₂.H₂O); copper carbonate basic (CuCO₃.Cu(OH)₂); cupric hydroxide (Cu(OH)₂); cuprous iodide (Cu₂I₂); cupric oxide (CuO); cuprous oxide (Cu₂O); and cupric chloride (CuCl₂). Typically, the antimicrobial copper salt is commercially available or can be prepared by art-recognized procedures from known compounds or intermediates readily available or prepared by known reaction schemes.

Preferably, the antimicrobial copper salt is cupric sulfate. Cupric sulfate occurs in the form of blue triclinic crystals (blue vitriol) and is a germicide/bactericide/fungicide.

More preferably, the antimicrobial agent is a mixture of cupric sulfate and aluminum chlorohydrate.

The antiviral agents in the present invention may be selected from a wide variety of water-soluble and water-insoluble drugs and their acid addition or metallic salts. Both organic and inorganic salts may be used provided the antiviral agent maintains its medicament value. The antiviral agents may be selected from a wide range of therapeutic agents and mixtures of therapeutic agents which may be administered in sustained release or prolonged action form. Nonlimiting illustrative categories and specific examples of such antiviral agents include the following medicaments: Acyclovir (9-[(2-hydroxyethyloxy)methyl]guanine); Foscarnet sodium (phosphonoformic acid trisodium salt); Ribavirin (1-beta-D-ribofuranosyl-1,2,4-triazole-3-carboxamide); Vidarabine (9-β-D-arabinofuranosyladenine monohydrate); Ganeiclovir sodium (9-(1,3-dihydroxy-2-propoxymethyl)guanine monosodium salt); Zidovudine [azidothymidine (AZT), 3′-azido-3′-deoxythymidine]; Phenol (carbolic acid); Amantadine hydrochloride (1-adamantanamine hydrochloride); and Interferon alfa-n3 (human leukocyte derived).

Preferred antiviral agents to be employed may be selected from the group consisting of acyclovir, foscarnet sodium, ribavirin, vidarabine, ganeiclovir sodium, zidovudine, phenol, amantadine hydrochloride, and interferon alfa-n3. In a preferred embodiment, the antiviral agent is selected from the group consisting of acyclovir, foscarnet sodium, ribavirin, vidarabine, and ganeiclovir sodium. In a more preferred embodiment, the antiviral agent is acyclovir.

The antibacterial agents which may be employed in the present invention may be selected from a wide variety of water-soluble and water-insoluble drugs and their acid addition or metallic salts. Both organic and inorganic salts may be used provided the antibacterial agent maintains its medicament value. The antibacterial agents may be selected from a wide range of therapeutic agents and mixtures of therapeutic agents which may be administered in sustained release or prolonged action form. Nonlimiting illustrative specific examples of antibacterial agents include bismuth containing compounds, sulfonamides, nitrofurans, metronidazole, tinidazole, nimorazole, benzoic acid, aminoglycosides, macrolides, penicillins, polypeptides, tetracyclines, cephalosporins, chloramphenicol, and clidamycin. Preferably, the antibacterial agent is selected from the group consisting of bismuth containing compounds, such as, without limitation, bismuth aluminate, bismuth subcitrate, bismuth subgalate, bismuth subsalicylate, and mixtures thereof; the sulfonamides; the nitrofurans, such as nitrofurazone, nitrofurantoin, and furozolidone; and miscellaneous antibacterials such as metronidazole, tinidazole, nimorazole, and benzoic acid; and antibiotics, including the aminoglycosides, such as gentamycin, neomycin, kanamycin, and streptomycin; the macrolides, such as erythromycin, clindamycin, and rifamycin; the penicillins, such as penicillin G, penicillin V, Ampicillin and amoxicillin; the polypeptides, such as bacitracin and polymyxin; the tetracyclines, such as tetracycline, chlorotetracycline, oxytetracycline, and doxycycline; the cephalosporins, such as cephalexin and cephalothin; and miscellaneous antibiotics, such as chloramphenicol, and clidamycin. More preferably, the antibacterial agent is selected from the group consisting of bismuth aluminate, bismuth subcitrate, bismuth subgalate, bismuth subsalicylate, sulfonamides, nitrofurazone, nitrofurantoin, furozolidone, metronidazole, tinidazole, nimorazole, benzoic acid, gentamycin, neomycin, kanamycin, streptomycin, erythromycin, clindamycin, rifamycin, penicillin G, penicillin V, Ampicillin amoxicillin, bacitracin, polymyxin, tetracycline, chlorotetracycline, oxytetracycline, doxycycline, cephalexin, cephalothin, chloramphenicol, and clidamycin.

The antimycotic agents which may be employed in the present invention may be selected from a wide variety of water-soluble and water-insoluble drugs and their acid addition or metallic salts. Both organic and inorganic salts may be used provided the antimycotic agent maintains its medicament value. The antimycotic agents may be selected from a wide range of therapeutic agents and mixtures of therapeutic agents which may be administered in sustained release or prolonged action form. Nonlimiting illustrative specific examples of antimycotic agents include heavy-metal fungicides, as well as organic fungicides and systemic fungicides; quinones such as chloranil and dichlone; organic sulfur compounds such as dithiocarbamates, chlorinated and nitrated benzenes; dithiocarbamates such as tetramethylthiuram disulfide, disodium ethylenebisdithiocarbamate, NaSSCNH(CH2)2-NHCSSNa, or nabam, and a manganese-stabilized material (maneb); imidazolines and guanidines such as heptadecyl-2-imidazolinium acetate (glyodin) which is synergistic with another fungicide, dodecylguanidinium acetate (dodine); trichloromethylthiocarboximides such as captan, (N-(trichloromethylthio)-4-cyclohexene-1,2-dicarboximide) folpet, (N-(trichloromethylthio)-phthalimide); chlorinated and nitrated benzenes such as 2,3,4,6-tetrachloronitrobenzene, pentachloronitrobenzene (PCNB), 1,2,4-trichloro-3,5-dinitrobenzene, 1,3,5-trichloro-2,4,6-trinitrobenzene, hexachlorobenzene, and tetrachloroisophthalonitrile; systemic fungicides such as oxathiins including 2,3-dihydro-5-carboxanilido-6-methyl-1,4-oxathiin (carboxin); benzimidazoles such as 2-(4-thiazoyl)benzimidazole and methyl (1-butylcarbamoyl)-2-benzimidazolylcarbamate (benomyl); pyrimidines such as 5-butyl-2-dimethylamino-6-methyl-4(1H)-pyrimidinone (dimethirimol) and 2-ethylamino analogue (ethirmol); and other fungicides such as dinoca fenaminosulf and the antimycotic antibiotic cycloheximide (actidone). Other antimycotic agents include partially halogenated hydantoins such as monochloro-5,5-dimethyl hydantoin (MCDMH), monobromo-5,5-dimethyl hydantoin (MBDMH), monochloro-5-methyl-5-ethyl hydantoin, monobromo-5-methyl-5-ethyl hydantoin, and combinations thereof. A most preferred hydantoin is monochlorodimethyl hydantoin.

The amount of antimicrobial agent used in the present invention is an antimicrobially effective amount and may vary depending upon the therapeutic dosage recommended or permitted for the particular antimicrobial agent. In general, the amount of antimicrobial agent present is the ordinary dosage required to obtain the desired result. Such dosages are known to the skilled practitioner in the medical arts and are not a part of the present invention. In a preferred embodiment, the antimicrobial agent is present in an amount from about 0.001% to about 5%, preferably from about 0.01% to about 5%, and more preferably from about 0.01% to about 3%, by weight.

In another embodiment, the present invention provides a method for preparing an antimicrobial fabric that exhibits antimicrobial properties comprising admixing an antimicrobially effective amount of an antimicrobial agent with a fibrous material.

The antimicrobial fabric can be prepared by a process involving treating or impregnating a fibrous material with the antimicrobial agent by any known method such as by capillary action (natural fabrics), non-capillary action (non-natural fabrics such as polyesters) including spraying or dipping or those methods used for sizing or scotch guarding. In general, the method for preparing an antimicrobial fabric may involve treating the fibrous material with the antimicrobial agent in a predetermined ratio in a detergent or laundry supplement. The detergent or laundry supplement may be in liquid form or powder form. The antimicrobial agent may be included in the powder detergent or laundry supplement and may also be supported on a porous material.

Detergents are generally composed of a surfactant along with optional additives. The additives contained in the detergent include builders, enzymes, fluorescent brighteners, anti-resoiling agents, bleaching agents, foam stabilizers, foam inhibitors, softening agents, solubilizers, thickeners, emulsifiers, perfumes, pigments, etc.

Suitable surfactants include anionic surfactants and non-ionic surfactants. The anionic surfactants may be alkylbenzenesulfonates, alpha-olefin-sulfonates, alkyl sulfates, polyoxyethylene alkyl sulfates, or salts of long-chain fatty acid. The non-ionic surfactants may be ether-type non-ionic surfactants, ether ester-type non-ionic surfactants, ester-type non-ionic surfactants, or nitrogen-containing non-ionic surfactants.

Suitable builders include water-soluble and water-insoluble inorganic and organic builders. Water-soluble builders may be phosphates such as sodium tripolyphosphate, trisodium phosphate, sodium metaphosphate, and sodium pyrophosphate; silicates such as sodium silicate; carbonates such as sodium carbonate, sodium bicarbonate, and sodium carbonate peroxyhydrate; sulfates such as sodium sulfate; and carboxylates such as sodium citrate. Water-insoluble builders may be crystalline alumino sodium silicate (zeolite), etc. Organic builders may be organic chelate builders, polymeric electrolytic builders, and organic activating builders. Organic chelate builders include aminocarboxylic acids such as nitrilotriacetic acid, ethylenediaminetetraacetic acid, and diethylenetriaminepentaacetic acid, and their salts; organic acids such as oxalic acid, tartaric acid, citric acid, gluconic acid, and their salts; cyclocarboxylic acid such as pyromellitic acid and benzopolycarboxylic acid, and their salts; and ether carboxylic acids, such as carboxymethyltartronic acid, carboxymethyloxysuccinic acid, and 2,5-dioxa-1,1,3,4,6,6-hexanehexacarboxylic acid. The polymeric electrolytic builder may be acrylic acid polymers, maleic anhydride polymers, co-polymers of acrylic acid and maleic anhydride, alpha-hydroxyacrylic acid polymers, itaconic acid polymers, and epoxysuccinic acid polymers; oxidized derivatives of natural polymers such as starch, cellulose, and alginic acid. The organic activating builder may be aminosulfonate.

The enzyme may be a protease, lipase, amylase, or cellulase, and the like.

The fluorescent brightener may be bis(triazinylamino)stilbene sulfonic acid, bisstyryl biphenyl, coumarin, pyrazoline, or naphthalimide, or a derivative thereof.

The anti-resoiling agent may be carboxymethyl cellulose, methyl cellulose, hydroxyethyl cellulose, carboxymethyl hydroxyethyl cellulose, hydroxypropyl cellulose, hydroxybutylmethyl cellulose, polyethyleneglycol, polyvinyl alcohol, polyvinyl pyrrolidone, polyvinyl acetate (vinyl acetate polymer), co-polymer of ethylene glycol and ethylene phthalate, co-polymer of vinyl pyrrolidone and vinyl acetate, co-polymer of vinyl sulfonic acid and sodium acrylate, co-polymer of vinyl acetate and maleic anhydride, co-polymer of vinyl pyrrolidone and maleic anhydride, co-polymer of vinyl sulfonic acid and maleic anhydride, and the like.

The bleaching agent may be sodium perborate, peroxyborax, sodium carbonate peroxyhydrate, sodium peroxypyrophosphate, perbenzoate, urea-hydrogen peroxide compound, melamine-hydrogen peroxide compound, citric acid perhydrate, and sodium perborate zinc phthalocyanine sulfonate, sodium perborate aluminium phthalocyanine sulfonate, and the like. The activating agent of the bleaching agent may be phthalic anhydride, benzoic anhydride, N,N′,N″,N″″-tetraacetylglycoluril, or tetraacetylethylenediamine. The stabilizing agent of the bleaching agent may be magnesium silicate, calcium silicate, tin silicate, and the like.

The foam stabilizer may be a diethanol-amide, a long-chain alcohol, amine oxide, carboxy betaine, sulfobetaine, hydroxyalkylamide, alkylsulfoxide, and the like.

The foam inhibitor may be a microcrystal wax, silicone, ketones with 18 to 40 carbon atoms, and the like.

The softening agent may be a cationic surfactant such as dimethylstearylammonium chloride, monoalkyldimethylamine oxide, high molecular weight polyamines, or montmorillonite, and the like.

Solubilizers may be benzenesulfonate, toluenesulfonate, xylenesulfonate, urea, ethanolamine, diethanolamine, triethanolamine, ethyl alcohol, ethylene glycol, propyleneglycol, polyethylene glycol, ethylbenzenesulfonic acid, isopropylbenzenesulfonate, cellosolve (ethyleneglycol monoethylether), and the like.

Thickeners may be maleic acid polymers, itaconic acid polymers, co-polymers of vinyl methyl ether and maleic anhydride, polypropylene glycols, polyvinyl alcohols, hydroxyethyl celluloses, methylhydroxypropyl celluloses, and hydroxypropyl celluloses; inorganic electrolytes such as sodium sulfide, sodium chloride, potassium chloride, and the like.

Emulsifiers may be co-polymers of styrene and acrylamido, co-polymers of styrene and vinylpyrrolidone, vinyl acetate polymers, magnesium stearate, monoglycerido, ethyleneglycol monofatty acid esters, ethyleneglycol difatty acid esters, argentine, micaceous titanium, and the like.

Anticorrosive and disinfectant agents may also be included such as dehydroacetic acid, sorbic acid, isobutyl p-hydroxybenzoate, isopropyl p-hydroxybenzoate, ethyl p-hydroxybenzoate, butyl p-hydroxybenzoate, n-propyl p-hydroxybenzoate, butylhydroxyanisole, butylhydroxytoluene, and salts thereof, and the like.

The types of perfumes and pigments are not particularly limited. A full recitation of all F.D.& C. perfumes and pigments and their corresponding chemical structures may be found in the Kirk-Othmer Encyclopedia of Chemical Technology, 3rd Edition, in volume 5 at pages 857-884, which text is incorporated herein by reference.

Laundry supplements are used before, during, or after washing for the purpose of enhancing the cleansing ability of the detergent or for the purpose of applying other treatment on the antimicrobial fabric.

The fibrous material (object to be treated/washed) is treated/washed in a solution containing the antimicrobial agent. The solution is preferably a cleansing solution containing the antimicrobial agent and a surfactant. This treatment allows the fibrous material to be subjected to the antimicrobial treatment as well as to be washed. The solution includes the antimicrobial agent in a cleansing solution containing the surfactant and/or laundry supplement. The method of adding the antimicrobial agent in the cleansing solution is not particularly limited as long as the sanitizing and antimicrobial effect is effected on the fibrous material (object to be treated/washed). Possible methods include adding the antimicrobial agent directly into the cleansing solution, spraying the antimicrobial agent onto the fibrous material in advance, soaking the fibrous material in advance in a liquid containing the antimicrobial agent, mixing the antimicrobial agent beforehand in the detergent or laundry supplement as a component thereof, and the like. Further, the antimicrobial agent may be added to the cleansing solution at any time between start and end of washing.

The concentration of the detergent and laundry supplement in accordance with the present invention in the cleansing solution is not particularly limited as long as it is within a range by which the disinfecting and antimicrobial properties are effected at the time of washing. However, in order to effect the antimicrobial properties sufficiently, the antimicrobial agent concentration of not less than 0.01 ppm in the cleansing solution is preferable. Further, when washing the same washing target object repeatedly, the antimicrobial effect can be obtained at lower concentrations.

Throughout this disclosure, applicant will suggest various theories or mechanisms by which applicant believes the components in the antimicrobial fabric exhibit antimicrobial properties in an unexpected manner. While applicant may offer various mechanisms to explain the present invention, applicant does not wish to be bound by theory. These theories are suggested to better understand the present invention but are not intended to limit the effective scope of the claims.

The apparatus useful in accordance with the present invention comprises apparatus well known in the manufacturing arts, and therefore the selection of the specific apparatus will be apparent to the artisan.

Throughout this application, various publications have been referenced. The disclosures in these publications are incorporated herein by reference in order to more fully describe the state of the art.

The invention being thus described, it will be obvious that the same may be varied in many ways. Such variations are not to be regarded as a departure from the spirit and scope of the invention and all such modifications are intended to be included within the scope of the following claims.

I claim:

1. An antimicrobial fabric comprising a fibrous material treated with an antimicrobially effective amount of an antimicrobial agent.

2. The antimicrobial fabric according to claim 1, wherein the fibrous material is a woven or a non-woven material.

3. The antimicrobial fabric according to claim 1, wherein the fibrous material is a disposable or a non-disposable material.

4. The antimicrobial fabric according to claim 1, wherein the fibrous material is a natural fiber selected from the group consisting of cotton, wool, silk, flax, mohair, linen, hemp, ramie, and jute.

5. The antimicrobial fabric according to claim 1, wherein the fibrous material is a non-natural fiber selected from the group consisting of rayons, acetates, nylons, polyesters, polyolefins, polyamides, acrylics, modacrylics fibers, fluorocarbons, glass fibers, and spandex.

6. The antimicrobial fabric according to claim 1, wherein the antimicrobial agent is selected from the group consisting of anti-viral agents, antibacterial agents, antimycotic agents, and combinations thereof.

7. The antimicrobial fabric according to claim 6, wherein the anti-viral agent is selected from the group consisting of acyclovir, foscarnet sodium, ribavirin, vidarabine, ganeiclovir sodium, zidovudine, phenol, amantadine hydrochloride, and interferon alfa-n3.

8. The antimicrobial fabric according to claim 6, wherein the antibacterial agent is selected from the group consisting of bismuth containing compounds, sulfonamides, nitrofurans, metronidazole, tinidazole, nimorazole, benzoic acid, aminoglycosides, macrolides, penicillins, polypeptides, tetracyclines, cephalosporins, chloramphenicol, and clidamycin.

9. The antimicrobial fabric according to claim 6, wherein the antimycotic agent is selected from the group consisting of heavy-metal fungicides, quinones, dithiocarbamates, imidazolines, guanidines, trichloromethylthiocarboximides, chlorinated and nitrated benzenes, (N-(trichloromethylthio)-4-cyclohexene-1,2-dicarboximide), (N-(trichloromethylthio)-phthalimide), oxathiins, benzimidazoles, pyrimidines, and partially halogenated hydantoins.

10. The antimicrobial fabric according to claim 1, wherein the antimicrobial agent is aluminum chlorohydrate, an aluminum chlorohydrate-containing compound, or an equivalent thereof.

11. The antimicrobial fabric according to claim 10, wherein the antimicrobial agent is selected from the group consisting of aluminum chlorohydrate, aluminum sesquicholorhydrate, aluminum dichlorohydrate, aluminum zirconium octachlorohydrate, aluminum zirconium pentachlorohydrate, aluminum zirconium tetrachlorohydrate, and aluminum zirconium trichlorohydrate.

12. The antimicrobial fabric according to claim 11, wherein the antimicrobial agent is aluminum chlorohydrate.

13. The antimicrobial fabric according to claim 1, wherein the antimicrobial agent is an antimicrobial copper salt.

14. The antimicrobial fabric according to claim 13, wherein the antimicrobial agent is selected from the group consisting of cupric sulfate, cupric acetate, copper carbonate basic, cupric hydroxide, cuprous iodide, cupric oxide, cuprous oxide, and cupric chloride.

15. The antimicrobial fabric according to claim 14, wherein the antimicrobial agent is cupric sulfate.

16. The antimicrobial fabric according to claim 1, wherein the antimicrobial fabric is a medical fabric selected from the group consisting of surgical gowns, scrubs, tabards, gloves, socks, hats, belts, shoes, insoles for shoes, padding, bandages, draping, sheeting, pillow cases, towels, and rugs.

17. The antimicrobial fabric according to claim 1, wherein the antimicrobial agent also acts as an indicator to distinguish between fibrous material treated with the antimicrobial agent and fibrous material untreated with the antimicrobial agent.

18. The antimicrobial fabric according to claim 1, wherein the fibrous material treated with the antimicrobial agent is distinguishable from fibrous material untreated with an antimicrobial agent by addition of an indicator to show treatment.

19. A method for preparing an antimicrobial fabric that exhibits antimicrobial properties comprising admixing an antimicrobially effective amount of an antimicrobial agent with a fibrous material.

20. The method according to claim 19, wherein the fibrous material is a woven or a non-woven material or a disposable or a non-disposable material.

21. The method according to claim 19, wherein the fibrous material is a natural fiber selected from the group consisting of cotton, wool, silk, flax, mohair, linen, hemp, ramie, and jute.

22. The method according to claim 19, wherein the fibrous material is a non-natural fiber selected from the group consisting of rayons, acetates, nylons, polyesters, polyolefins, polyamides, acrylics, modacrylics fibers, fluorocarbons, glass fibers, and spandex.

23. The method according to claim 19, wherein the antimicrobial agent is selected from the group consisting of anti-viral agents, antibacterial agents, and antimycotic agents.

24. The method according to claim 19, wherein the antimicrobial agent is aluminum chlorohydrate, an aluminum chlorohydrate-containing compound, or an equivalent thereof.

25. The method according to claim 24, wherein the antimicrobial agent is selected from the group consisting of aluminum chlorohydrate, aluminum sesquicholorhydrate, aluminum dichlorohydrate, aluminum zirconium octachlorohydrate, aluminum zirconium pentachlorohydrate, aluminum zirconium tetrachlorohydrate, and aluminum zirconium trichlorohydrate.

26. The method according to claim 25, wherein the antimicrobial agent is aluminum chlorohydrate.

27. The method according to claim 19, wherein the antimicrobial agent is an antimicrobial copper salt.

28. The method according to claim 27, wherein the antimicrobial agent is selected from the group consisting of cupric sulfate, cupric acetate, copper carbonate basic, cupric hydroxide, cuprous iodide, cupric oxide, cuprous oxide, and cupric chloride.

29. The method according to claim 28, wherein the antimicrobial agent is cupric sulfate.

30. The method according to claim 19, wherein the antimicrobial fabric is a medical fabric selected from the group consisting of surgical gowns, scrubs, tabards, gloves, socks, hats, belts, shoes, insoles for shoes, padding, bandages, draping, sheeting, pillow cases, towels, and rugs.

31. The method according to claim 19, wherein the antimicrobial agent also acts as an indicator to distinguish between fibrous material treated with the antimicrobial agent and fibrous material untreated with the antimicrobial agent.

32. The method according to claim 19, wherein the fibrous material treated with the antimicrobial agent is distinguishable from fibrous material untreated with an antimicrobial agent by addition of an indicator to show treatment.
